(19) United States
(12) Patent Application Publication
(10) Pub. No.: US 2018/0243207 A1
(43) Pub. Date: Aug. 30, 2018

(54) DUAL-RELEASE FLEXIBLE FORMULATION USEFUL IN PALLIATIVE AND HOSPICE CARES OF ELDERLY PATIENTS

(71) Applicant: PADMAKSHI SINGH, JERSEY CITY, NJ (US)

(72) Inventor: PADMAKSHI SINGH, JERSEY CITY, NJ (US)

(21) Appl. No.: 15/793,033

(22) Filed: Oct. 25, 2017

Related U.S. Application Data
(60) Provisional application No. 62/464,118, filed on Feb. 27, 2017.

(51) Int. Cl. A61K 9/00; A61K 31/5513; A61K 47/32; A61K 47/34; A61K 47/36

(52) U.S. Cl. CPC A61K 9/006; A61K 31/5513; A61K 47/36; A61K 47/34; A61K 47/32

(57) ABSTRACT

Described herein is a single-construct of film that provides a dual (both a rapid and sustained)-release, dual-adhesiveness flexible thin film formulation for a class of benzodiazepines and a method of administration of the benzodiazepines by using the thin film formulation which is suitable for application to the oral cavity (buccal site or on the tongue).

### CLAIM OF PRIORITY

This application claims priority to U.S. Application 62/464,118 filed on Feb. 27, 2017, the contents of which are herein fully incorporated by reference in its entirety.

### FIELD OF THE EMBODIMENTS

The embodiments of the present invention relate to the field of convenient dual-release flexible formulations for benzodiazepine family drugs, including lorazepam, primarily adapted to use in the mucosal tissue for providing a rapid and a sustained release of the benzodiazepine drugs for various applications in elderly patients in palliative, hospice and other cares.

### BACKGROUND OF THE EMBODIMENTS

According to at least one study (Tradounsky, G. Seizures in Palliative Care, Canadian Family Physician/Le Médecin de famille canadien (2013) 59(9): 951-955), in the contexts of palliative and hospices cares, seizures can occur in about 13% or more of cases. (Grewal J, Grewal H K, Forman A D. Seizures and epilepsy in cancer: etiologies, evaluation, and management. Curr Oncol Rep (2008) 10(1): 63-71 and Singh G, Rees J H, Sander J W., Seizures and epilepsy in oncological practice: causes, course, mechanisms and treatment. J Neurol Neurosurg Psychiatry (2007) 78(4): 342-9). About 25% to 50% of palliative patients who develop seizure activity have brain metastases. (Singh G, Rees J H, Sander J W., Seizures and epilepsy in oncological practice: causes, course, mechanisms and treatment. J Neurol Neurosurg Psychiatry (2007) 78(4): 342-9 and Beaulieu I, Nadeau C., Myoclonies et convulsions. In: Beausoleil M, Association des pharmaciens des établissements de santé du Québec. Guide pratique des soins palliatifs: gestion de la douleur et autres symptômes. 4th Ed. Montreal, QC: Association des pharmaciens des établissements de santé du Québec; 2008. p. 287-98). Of patients with primary brain tumors, 20% to 45% will present at diagnosis with convulsions and more will develop seizures as their cancer progresses. Other common end-of-life (EOL) symptoms include agitation and delirium, anxiety, fatigue, insomnia, weakness, constipation, nausea, and vomiting. Medications for Terminal Restlessness and Agitation include anti-anxiety medications such as Lorazepam (marketed as Ativan) and Diazepam (Valium) and anti-psychotic medications such as Haloperidol (Haldol), Chlorpromazine HCl (Thorazine) and others.

According to another study (Stiel, S. et al., Indikationen and Gebrauch von Benzodiaze-pinen auf einer Palliativstation, Schmerz. (2008) 22(6): 665-71), benzodiazepines are often used in palliative care for symptom control, for example in treatment of dyspnea or anxiety. They are also used for palliative sedation, if symptoms are not controlled (fast) enough and a reduction of consciousness is therefore necessary. Incidence of palliative sedation has been reported to vary between 5% to about 52% of all patients in a palliative care setting in several studies. The study results indicated that of the patients 70% were treated with benzodiazepines but only 3.1% received deep and continuous sedation. Benzodiazepines were applied as required in oral or sublingual form in 71.8% of all cases. Amongst the benzodiazepines, lorazepam was given most often because of anxiety and agitation, oxazepam to induce sleep and midazolam in five cases of terminal sedation. Only rarely were diazepam and clonazepam prescribed to prevent or resolve convulsions. For example, Lorazepam sublingually 0.5 mg, is typically given as required for episodic anxiety, panic attacks.

As described above, Lorazepam is a common hospice medication and is used to reduce anxiety and/or restlessness. It may also be used for shortness of breath and insomnia. It is most effective when taken before symptoms become severe and is generally started at the lowest dose and increased if needed to treat symptoms. The most common side effect of Lorazepam is sedation. It is acceptable to give both Lorazepam and morphine at the same time if needed for comfort. In general, Lorazepam tablets can be placed under the tongue or in the cheek if the patient cannot swallow. The tablets can also be dissolved in a small amount of warm water and placed under the tongue or in the cheek.

Conventional oral formulations, such as tablets, pills, caplets, or capsules, are designed for short residence time in the mouth. Absorption of the active pharmaceutical ingredient (API) from these formulations occurs in the gastrointestinal (GI) tract only after the dosage form undergoes disintegration followed by dissolution of the agent in the gastric fluids. These formulations are thus not suitable for delivering doses of benzodiazepines, in general, and Lorazepam, in particular, for various applications of such drugs in elderly patients in palliative, hospice and other cares, where patients can experience convulsions, or otherwise have difficulties to swallow and thus are less likely to be compliant with conventional oral dosing.

Recently, new advanced formulations, i.e., mucoadhesive films (also referred to as orally dissolving or eroding thin films or oral bioadhesive films) that provide a useful alternative to traditional drug formulations. They are typically designed for intra-oral administration by patients placing the strip on or under the tongue (lingual or sublingual) or along the inside of the cheek (buccal). They are made similar in size, shape and thickness to a postage stamp (as thin film strips, squares or discs, containing API), and as such thin films dissolve/erode, API is released and delivered to the blood stream either intragastrically, buccally or sublingually.

Based on their dissolving speeds, mucoadhesive films can generally be classified into two categories: fast dissolving films and slow dissolving/eroding films. The former films typically comprise polymers of high water solubility and are designed as a convenient form for lingual administration and gastro-intestinal tract absorption. The active ingredients are incorporated in the film matrix, which dissolves rapidly on the tongue and is then swallowed into the GI tract for absorption. As no water is required, this dosage form is convenient for the consumer or patient. The latter (slow dissolving) films are mainly designed for systemic administration via the interior lining of the cheek (buccal mucosa) or for local treatment.

Additional examples of related art in the field are described below:

U.S. Patent Application No. 2011/0237563 relates to film formulations that are specially formulated to meet exacting bioavailability requirements, or to be bioequivalent to existing orally administered formulations and which is particularly suitable for patients with dysphagia as they quickly disintegrate in the mouth when exposed to saliva, and does not need added water to accelerate its disintegration. These film formulations are non-mucoadhesive, and are absorbed predominantly through the gastrointestinal tract, as they are formulated or administered for gastrointestinal absorption of the bioactive agent.

U.S. Patent Application No. 2013/0039932 relates to a quickly soluble oral film dosage for masking a nasty taste. A quickly soluble oral film dosage comprises a stevioside based sweetener and a high potency sweetener in a ratio of 1:3 to 3:1 (w/w), which may efficiently mask a bitter or nasty taste of a medicine and may be quickly dissolved in a mouth without water, thereby improving an aftertaste thereof and enhancing dosage acceptability of a patient.

U.S. Patent Application No. 2015/0038540 relates to an instantly wettable and rapidly disintegrating oral film dosage form without a surfactant and without a polyalcohol, which was achieved by combining at least one water soluble polymer that is not a copolymer of vinylpyrrolidone, at least one active agent, a copolymer of vinylpyrrolidone and titanium dioxide. In certain embodiments, the film comprises hydroxypropyl cellulose or a combination of hydroxypropyl cellulose and a polymer or copolymer of vinylpyrrolidone or a substituted vinylpyrrolidone as the water soluble polymer(s). A plasticizer, and optional additives selected from synthetic sweeteners, natural sweeteners, flavorants, antioxidants, colorants, and opacifiers, can be added to the disclosed film oral formulations.

International Application No. PCT/EP2007/008579 (publication No. WO2008/040534) relates to orally disintegrating film formulations for delivering active pharmaceutical agents, methods of formulating the formulations to retard absorption through the oral mucosa, and methods of using the formulations for the treatment of various medical conditions.

None of the art described above can address all of the issues that the present invention does, because, regrettably, the aforementioned formulations have proven futile in palliative, hospice and other settings, where patients can experience convulsions, or otherwise have difficulties to swallow and thus are less likely to be compliant with conventional oral dosing. As a result, there is an unmet need in the art for alternative and more effective while lasting delivery of benzodiazepines/Lorazepam for palliative and hospice medication. The present invention relates to combining the aforementioned advantages of both fast dissolving films and slow eroding films to arrive at a bilayer film construct suitable for delivering benzodiazepines/Lorazepam in palliative, hospice and the like. The construct can provide for ease of application.

### SUMMARY OF THE EMBODIMENTS

In certain embodiments, the present invention provides a dual-release, dual-adhesiveness flexible thin film formulation, in the form of a single-construct of film that provides both a rapid and sustained release of a class of benzodiazepines. Another aspect of the present invention is the innovative method of administration of the class of benzodiazepines involved. In certain embodiments of the present invention, the thin film formulation is applied to the oral cavity (buccal site or on the tongue). The film is designed such that it disintegrates and dissolves upon administration and the benzodiazepine dosage is released for oral and/or transmucosal absorption. In addition, certain aspect of the present invention also can provide a method to alleviate or eliminate undesired taste or sensation of benzodiazepines in the oral thin film.

In one of the embodiments, the present invention provides, inter alia, a dual-release, dual-adhesiveness flexible thin film formulation for delivering a class of benzodiazepines that comprise a quick-release subfilm of an admixture of polymer (predominantly highly water-soluble polymers), and a benzodiazepine in a first form, the polymers and other components of the quick-release subfilm adapted to provide 20 minute or less erodibility for the quick-release subfilm; and against said quick-release subfilm is laminated a sustained-release subfilm comprising an admixture of polymer, which comprises predominantly hydrophilic, bioadhesive polymer(s), and said benzodiazepine in a second form (can be the same as the first), the polymers and other components of the sustained-release subfilm adapted to provide erodibility of between 1 hours or more and 20 hours or less for the sustained-release subfilm. The resulting thin film formulation of said benzodiazepine(s) has a thickness of 30 mil or less, and is more adhesive on the sustained-release side.

In one of the aspects of the embodiment, the highly water-soluble polymers in the aforementioned thin film formulation of the present invention comprises soluble cellulose derivatives, polyvinylpyrrolidone, polyvinyl alcohol, polyethylene oxide, polyvinyl alcohol grafted polyethylene glycol copolymer, or mixtures thereof.

In another one of the aspects of the embodiment, the highly water-soluble polymers in the aforementioned thin film formulation of the present invention comprises soluble polyethylene oxide, polyvinyl alcohol grafted polyethylene glycol copolymer, or mixtures thereof.

In yet another one of the aspects of the embodiment, the predominantly hydrophilic, bioadhesive polymers in the aforementioned thin film formulation of the present invention comprise xanthan gum, carrageenan, pectin, sodium carboxymethylcellulose, alginate, polyacrylic acids, high molecular weight polyethylene oxide, or mixtures thereof.

In still another one of the aspects of the embodiment, the predominantly hydrophilic, bioadhesive polymers in the aforementioned thin film formulation of the present invention comprise polyacrylic acids, high molecular weight polyethylene oxide, or mixtures thereof.

In still another one of the aspects of the embodiment, the polymers and other components of said quick-release subfilm adapted to provide 15 minute or less erodibility for the quick-release subfilm.

In yet still another one of the aspects of the embodiment, the polymers and other components of said quick-release subfilm are adapted to provide 10 minute or less erodibility for the quick-release subfilm.

In yet still another one of the aspects of the embodiment, the polymers and other components of said sustained-release subfilm are adapted to provide bioactive agent release period of 2 hours or more and 20 hours or less for the bioactive agent provided by the sustained-release subfilm.

In another one of the embodiments, the benzodiazepine to be released from the thin film formulation of the present invention is a compound belonging to the benzodiazepine class of a sub-unit selective positive allosteric modulator (PAM) of the GABAA receptor in the central nervous system (CNS) and is selected from the group consisting of lorazepam, diazepam, alprazolam, estazolam, temazepam, chlordiazepoxide, clonazepam, diazepam, flurazepam, quazepam, clorazepate, oxazepam, midazolam, and triazolam or pharmaceutically acceptable salts thereof.

In yet another one of the embodiments, the present invention provides a method of delivering a benzodiazepine that functions as sub-unit selective PAM of the GABAA receptor in the CNS, which comprises applying the sustained-release side of the thin film formulation of the present invention to the mucosal tissue of an elderly subject in need of palliative, hospice and other cares.

In one of the aspects of the aforementioned method embodiment, the benzodiazepine formulation is applied to the mucosal tissue of the subject's mouth, wherein the quick-release side of the benzodiazepine formulation is applied to the top of the tongue, and the formulation is applied by a transfer from the tongue to the palate of the subject.

In one of the aspects of the embodiment, the method of the present invention further comprises providing the benzodiazepine formulation so that the quick-release and the sustained release sides have distinct colors, applying the quick-release colored side to a transfer intermediate, and then transferring the formulation to the mucosal tissue of the subject.

In yet another one of the embodiments, the present invention provides, inter alia, a flexible thin film formulation of a benzodiazepine consisting of a sustained-release film which comprises: i) a benzodiazepine; ii) an admixture of polymer to be admixed with said benzodiazepine, which comprises predominantly hydrophilic, bioadhesive polymer(s) and polymers and other components of the sustained-release subfilm adapted to provide a bioactive release period of 1.5 hours or more and 15 hours or less, wherein the thin film formulation of the benzodiazepine having a thickness of 30 mil or less, and wherein a predominant amount of the predominantly hydrophilic bioadhesive polymer(s) comprises cross-linked polymers of acrylic acid.

In one of the aspects of the embodiment, the predominantly hydrophilic, bioadhesive polymers comprise polyacrylic acids, high molecular weight polyethylene oxide, or mixtures thereof.

In another one of the aspects of the embodiment, the benzodiazepine is a sub-unit selective positive allosteric modulator (PAM) of the GABAA receptor in the central nervous system (CNS) and is selected from the group consisting of consisting of lorazepam, diazepam, alprazolam, estazolam, temazepam, chlordiazepoxide, clonazepam, diazepam, flurazepam, quazepam, clorazepate, oxazepam, midazolam, and triazolam or pharmaceutically acceptable salts thereof.

In yet another one of the embodiments, the present invention provides a dual-release, dual-adhesiveness flexible thin film formulation for delivering a class of benzodiazepines, wherein the predominantly hydrophilic, bioadhesive polymers in the admixture of polymer of the quick-release subfilm comprise xanthan gum, carrageenan, pectin, sodium carboxymethylcellulose, alginate, polyacrylic acids, high molecular weight polyethylene oxide, or mixtures thereof.

In still yet another one of the embodiments, the present invention provides a dual-release, dual-adhesiveness flexible thin film formulation for delivering a class of benzodiazepines, wherein the predominantly hydrophilic, bioadhesive polymers in the admixture of polymer of the sustained-release subfilm comprise xanthan gum, carrageenan, pectin, sodium carboxymethylcellulose, alginate, polyacrylic acids, high molecular weight polyethylene oxide, or mixtures thereof.

In still yet another one of the embodiments, the present invention provides a dual-release, dual-adhesiveness flexible thin film formulation for delivering a class of benzodiazepines, wherein the polymers and other components of the quick-release subfilm adapted to provide 15 minute or less erodibility for the quick-release subfilm.

In still yet another one of the embodiments, the present invention provides a dual-release, dual-adhesiveness flexible thin film formulation for delivering a class of benzodiazepines, wherein the polymers and other components of the quick-release subfilm adapted to provide 15 minute or less erodibility for the quick-release subfilm.

In still yet another one of the embodiments, the present invention provides a dual-release, dual-adhesiveness flexible thin film formulation for delivering a class of benzodiazepines, wherein the polymers and other components of the quick-release subfilm adapted to provide 15 minute or less erodibility for the quick-release subfilm.

In still yet another one of the embodiments, the present invention provides a method to treat an elderly subject in need of a single dose of a benzodiazepine in a dual-release formulation comprises applying the sustained-release side of the dual-adhesiveness flexible thin film formulation of the present invention to the mucosal tissue of the subject.

In one of the aspects of the aforementioned method embodiment, the benzodiazepine is a sub-unit selective positive allosteric modulator (PAM) of the GABAA receptor in the central nervous system (CNS) and is selected from the group consisting of lorazepam, diazepam, alprazolam, estazolam, temazepam, chlordiazepoxide, clonazepam, diazepam, flurazepam, quazepam, clorazepate, oxazepam, midazolam, and triazolam or pharmaceutically acceptable salts thereof.

In another one of the aspects of the aforementioned method embodiment, the elderly subject who can benefit from a single dose of a benzodiazepine in a dual-release formulation as a sub-unit selective PAM of the GABAA receptor in the CNS is likely suffering from one or more of the conditions relating to the subject's old age, which condition is selected from the group consisting of dyspnea (shortness of breath), respiration congestion, agitated delirium, pain (e.g., pleuritic pain, discomfort from immobility and/or pain due to pre-existing conditions), weakness and fatigue, insomnia, nausea and vomiting, seizures and poor secretion control.

In still yet another one of the embodiments, the present invention provides a method to treat an elderly subject in need of a single dose of a benzodiazepine in a sustained-release formulation comprises applying the sustained-release flexible thin film formulation of the present invention to the mucosal tissue of the subject.

In one of the aspects of the aforementioned method embodiment, the benzodiazepine is a sub-unit selective positive allosteric modulator (PAM) of the GABAA receptor in the central nervous system (CNS) and is selected from the group consisting of lorazepam, diazepam, alprazolam, estazolam, temazepam, chlordiazepoxide, clonazepam, diazepam, flurazepam, quazepam, clorazepate, oxazepam, midazolam, and triazolam or pharmaceutically acceptable salts thereof.

In another one of the aspects of the aforementioned method embodiment, the elderly subject who can benefit from a single dose of a benzodiazepine in a sustained-release formulation as a sub-unit selective PAM of the GABAA receptor in the CNS is likely suffering from one or more of the conditions relating to the subject's old age, which condition is selected from the group consisting of dyspnea (shortness of breath), respiration congestion, agitated delirium, pain (e.g., pleuritic pain, discomfort from immobility and/or pain due to pre-existing conditions), weakness and fatigue, insomnia, nausea and vomiting, seizures and poor secretion control.

It is an objective of the present invention to provide a dual-release, dual-adhesiveness flexible thin film formulation, in the form of a single-construct of film, for both a rapid and sustained release of a class of benzodiazepines.

It is another objective of the present invention to provide an innovative method of administration of said class of benzodiazepines for an elderly subject in need of palliative, hospice and other cares.

It is yet another objective of the present invention provides a method to treat an elderly subject in need of a single dose of a benzodiazepine in a dual-release formulation by applying the sustained-release side of the dual-adhesiveness flexible thin film formulation of the present invention to the mucosal tissue of the subject. The film is designed such that it disintegrates and dissolves upon administration and the benzodiazepine dosage is released for oral and/or transmucosal absorption.

It is yet still another objective of the present invention that the dual-release thin film formulation is applied to the oral cavity (buccal site or on the tongue) of the elderly subject in need of palliative, hospice and other cares.

It is yet still another objective of the present invention to provide a method to alleviate or eliminate undesired taste or sensation of benzodiazepines in the dual-release oral thin film.

It is yet still another objective of the present invention that the benzodiazepine being delivered is a sub-unit selective positive allosteric modulator (PAM) of the GABAA receptor in the central nervous system (CNS) and is selected from the group consisting of lorazepam, diazepam, alprazolam, estazolam, temazepam, chlordiazepoxide, clonazepam, diazepam, flurazepam, quazepam, clorazepate, oxazepam, midazolam, and triazolam or pharmaceutically acceptable salts thereof. The elderly subject who can benefit from such a single dose of a benzodiazepine in a dual-release formulation is likely suffering from one or more of the conditions relating to the subject's old age, which condition is selected from the group consisting of dyspnea (shortness of breath), respiration congestion, agitated delirium, pain (e.g., pleuritic pain, discomfort from immobility and/or pain due to pre-existing conditions), weakness and fatigue, insomnia, nausea and vomiting, seizures and poor secretion control.

### BRIEF DESCRIPTION OF THE DRAWINGS

FIG. 1 shows drug release profile of 0.5 mg lorazepam oral film.

FIG. 2 shows drug release profile of 2 mg lorazepam oral film.

FIG. 3 shows drug release profile of 1 mg slow releasing oral film from Example 3.

FIG. 4 shows drug release profiles for individual QD and SL films.

FIG. 5 shows drug release profiles of combined (lamination) of 2-layered matrix film.

FIG. 6A shows a first view of an embodiment of the present invention.

FIG. 6B shows a second view of an embodiment of the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

### Definition

Numerical ranges, as used herein, are intended to include sequential integers. For example, a range expressed as “10-20%” would include 10%, 11%, 12%, 13%, 14%, 15%, 16%, 17%, 18%, 19% and 20%. Similarly, numerical ranges are also intended to include sequential fractional integers. For example, a range expressed as “1-2%” would include 1.0%, 1.1%, 1.2%, 1.3%, 1.4%, 1.5%, 1.6%, 1.7%, 1.8%, 1.9% and 2.0%.

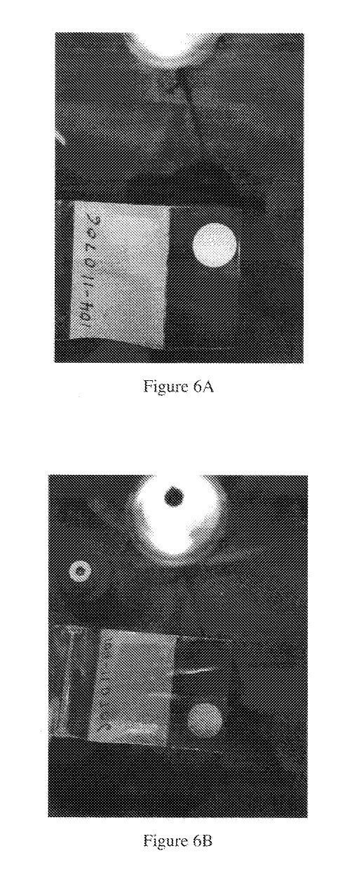

According to one of the embodiments, the formulations of the present invention for benzodiazepines, in general, and Lorazepam, in particular, are in the forms of dual-release films constructed from, at the least, two subfilms of substantially different erodibilities: one for quick-release and one for sustained-release. Each of the subfilms comprises, in addition to benzodiazepine (Lorazepam), which amounts for 0.1 to 30%, preferably, 0.1 to 15% and more preferably, 0.3-10% (Weight), inter alia, polymers, taste modifiers, plasticizers and or release modifiers, buffering agents and preservatives, as shown in the table below (see FIGS. 6A and 6B).

| | Quick-release | Sustained-release |
|---|---|---|
| Ingredient Class | subfilm (Wt. %) | subfilm (Wt. %) |
| Benzodiazepine | 0.1-30 | 0.1-30 |
| | (preferably 0.3-10) | (preferably 0.3-10) |
| Mucosal Bioadhesive | 0.1-10 | 15-40 |
| Polymer | | |
| Film Matrix Polymer | 50-85 | 0.1-25 |
| Plasticizer | 5 or 10 or 15-40 | 15-40 |
| Taster modifier | 0.1 or 3-10 or 12 | 3-10 or 12 |

According to one aspect of the embodiment, the substantial differences in the erodibility of said at least two subfilms arise from the different combinations of the types of polymers the two subfilms each comprises (as shown above). In other words, the faster erodibility of the quick-release film or subfilm is made possible by the presence of less than 10% of Mucosal Bioadhesive Polymers, which are predominantly hydrophilic, in combination with, 50-85% of Film Matrix Polymer that are highly water-soluble. On the other hand, the sustained-release film or subfilm, containing 15-40% of Mucosal Bioadhesive Polymers in combination of 0.1-25% of Film Matrix Polymer, has a relatively slower erodibility of the subfilm, thus leading to a sustained-release of the API, in this case, benzodiazepine in general and (Lorazepam in particular). According to another aspect of the embodiment, the ranges of the weight amounts of the ingredients in any film or subfilm, in addition to benzodiazepine (Lorazepam) of 0.1 to 30%, preferably, 0.1 to 15% and more preferably, 0.3-10%, can be:

Mucosal Bioadhesive Polymers: 0.1-45%, preferably 0.1-20% for quick release subfilm and 5-45% for sustained-release subfilm, and more preferably 0.1-10% for quick release subfilm and 15-40% for sustained-release subfilm;

Film Matrix Polymer: 0.1-90%, preferably 30-90% for quick release subfilm and 0.1-30% for sustained-release subfilm, and more preferably 50-85% for quick release subfilm and 0.1-25% for sustained-release subfilm;

Plasticizer/release agent: 1-50%, preferably 5-45% for quick release subfilm and 5-45% for sustained-release subfilm, and more preferably 10-40% for quick release subfilm and 15-40% for sustained-release subfilm;

Taste modifier: 1-20%, preferably 0.1-15% for quick release subfilm and 0.5-15% for sustained-release subfilm, and more preferably 0.5-12% for quick release subfilm and 3-12% for sustained-release subfilm;

Buffering agents: as needed or 0.1-10%, preferably 0.5-5% and more preferably 1-2%.

According to yet another aspect of the embodiment, Film Matrix Polymers which are highly water-soluble and thus, primarily responsible for the quick-releasing properties, include, inter alia, cellulose derivatives (e.g., hydroxypropyl methyl-cellulose), polyvinylpyrrolidone, polyvinyl alcohol, polyethylene oxide, and polyvinyl alcohol grafted polyethylene glycol copolymer (Kollicoat® IR, BASF), or mixtures thereof. For example, PolyOx™ WSR N-10 (Dow), a polyethylene oxide with MW 100,000, can be used.

According to still another aspect of the embodiment, Mucosal Bioadhesive Polymers that are predominantly hydrophilic include, inter alia, unmodified natural polymers, modified natural polymers, and synthetic polymers. For example, predominantly hydrophilic, bioadhesive polymers can be xanthan gum, carrageenan, pectin, sodium carboxymethylcellulose, alginate, polyacrylic acids, high molecular weight polyethylene oxide, or mixtures thereof.

In addition, the sustained-release subfilm can contain water-insoluble polymers such as ethyl cellulose, propyl cellulose, polyethylene, polypropylene or the like.

According to still another aspect of the embodiment, plasticizers, for example, propylene glycol, glycerin, PEG-4000, PEG-400, can provide flexibility to the formulations and their surfactant properties can act as a release modifier in affecting the API release profile. Taste modifiers such as flavors, sweeteners, and taste masking agents can be incorporated in the dosage form to provide a pleasant taste and mouth-feel when the dosage form is administered in the oral cavity.

Taste modifiers such as flavors, sweeteners, and taste masking agents can be incorporated in the dosage form to provide a pleasant taste and mouth-feel when the dosage form is administered in the oral cavity. Taste modifiers include, for example, flavoring agents (such as peppermint oil, other flavored oils, Cocoa powder, and the like), sweeteners (such as sodium saccharin, glucose, fructose, Aspartame, Sucralose, Steviosides, and the like), modifiers (such as Cremophor® RH-40 (polyoxy 40 hydrogenated castor oil, BASF), Clove oil, diglycerides), taste masking agents (such as Magnasweet 100 (mono-ammonium glycyrrhizinate, Mafco, Inc.), Eudragit E-100 (2-dimethylamino)ethyl methacrylate polymer, Evonik)), and the like.

According to still another aspect of the embodiment, preservatives or stabilizers can also be employed, as needed. Such preservatives can include, inter alia, anti-microbial agents and non-organic compounds, such as sodium benzoate, parabens and derivatives, ascorbic acid and salts, propionic acids and salts, sulfur dioxide and sulfites, acetic acid and acetates, nitrites and nitrates, and the like.

According to another of the embodiments, the film, or subfilm, formulations of benzodiazepines of the present invention can be stand-alone or self-supporting, meaning they should have enough integrity so that there is no need to support them with additional backings, such as with polyethylene films that are non-dissolving. The dosage form (either single or dual layer) of benzodiazepines of the present invention can be in a shape of square, rectangular, circular, oval, or any number of other shapes. The square formulations benzodiazepines of the present invention can be, for instance, 0.5-8 cm on the side, preferably 1-6 cm, more preferably 1-5 cm and even more preferably 1-4 cm. Circular (disk) formulations of benzodiazepines of the present invention can be, for instance, 0.5-8 cm in diameter, preferably 1-6 cm, more preferably 1-4 cm and even more preferably 1-2 cm.

According to one aspect of the embodiment, the dosage form (either single or dual layer) of benzodiazepines of the present invention can be, for example, 60 mil (1.524 mm) or less in thickness, preferably 40 mil (1.016 mm) or less, more preferably 30 mil (0.762 mm) or less and even more preferably 20 mil (0.508 mm) or less. The quick-release subfilm can be, for example, 30 mil (0.762 mm) or less, more preferably 20 mil (0.508 mm) or less and even more preferably 10 mil (0.254 mm) or less. The sustained-release subfilm can be, for example, 30 mil (0.762 mm) or less, more preferably 20 mil (0.508 mm) or less and even more preferably 10 mil (0.254 mm) or less.

According to another aspect of the embodiment, when needing higher dosages of benzodiazepines of the present invention, more than one dosage form can be used at each administration, such as 1-4 dosage forms per administration and the administrations can be repeated as appropriate. Dosages of benzodiazepines of the present invention may be for example from 0.01 mg per administration to 0.1 mg per administration; from 0.1 mg per administration to 1 mg per administration; from 1 mg per administration to 10 mg per administration; from 10 mg per administration to 100 mg per administration.

According to still yet another of the embodiments, the formulations of the present invention for benzodiazepines, in general, and Lorazepam, in particular, can exhibit one or more of the following characteristics:
1) The formulations have sufficient flexibility to adapt to the surface of the mucosal tissue to which it is adapted to be administered;
2) They are comfortable and unobtrusive during use;
3) They are easy to administer to the mucosal tissue;
4) They remain in place on the mucosal tissue without moving once administered;
5) They are capable of providing a rapid release of benzodiazepines immediately subsequent to administration followed by a sustained release of benzodiazepines for an extended period; and
6) They completely dissolve and/or erode at the end of the release period without the need for the physical removal of any residue.

According to another aspect of the embodiment, the formulations of the present invention for benzodiazepines, in general, and Lorazepam, in particular, are released and delivered to the blood stream via the gastrointestinal system or via mucosal tissue, or released and delivered locally at or near the site (i.e., the mucosal tissue) where the formulations are adhered. Typically, where the dosage form is used in the mouth, any transmucosal delivery may be, for example, buccal, sublingual, via the palate, or the like. Particularly for benzodiazepines from the sustained-release layer, it can be that some is delivered transmucosally at or near the site that the dosage form is adhered, and some is delivered via the gastrointestinal tract. Furthermore, the site of application for the formulations of the present invention are not limited to only the oral mucosal tissue. Other mucosal tissue, such as rectal or vaginal tissue, can also be used.

According to yet another aspect of the embodiment, the various polymers and other components of the quick-release subfilm in the formulations of the present invention for benzodiazepines, in general, and Lorazepam, in particular, are adapted to provide 60 minutes or less erodibility for the quick-release subfilm, preferably 40 minutes or less erodibility, more preferably 30 minutes or less erodibility and even more preferably 20 minutes or less erodibility.

According to yet another aspect of the embodiment, the various polymers and other components of the sustained-release subfilm in the formulations of the present invention for benzodiazepines, in general, and Lorazepam, in particular, are adapted to provide 30 hours or less erodibility for the sustained-release subfilm, preferably 20 hours or less erodibility, more preferably 10 hours or less erodibility and even more preferably 5 hours or less erodibility.

According to still yet another of the embodiments, the formulations of the present invention for benzodiazepines, in general, and Lorazepam, in particular, can be prepared, for example, by the solvent-casting method, or by the hot-melt extrusion process. Generally, solvent-based casting process for fabricating a film involves three basic steps: a) preparation of wet casting solution, b) casting of wet film, and c) drying of the film. There are generally three more steps for finishing of the device fabrication process: d) die-cutting of the film into individual unit-dose discs, e) de-lamination of thin-film discs from a casting liner, and f) pouching of the discs. The final thin-form discs can be individually packaged in single pouches as single unit doses.

While not wishing to be bound by theory, it is believed that as provided by the present invention, the benzodiazepine (Lorazepam) profoundly attenuates anxiety-related symptoms and also prevents prevent nausea and vomiting by preferentially acting on the GABA α1-, (as well as α2- and α3-) receptor subunit(s). Because of the relatively non-selective pharmacological profile of Lorazepam, which includes a high affinity to the α3-GABA_{A} receptors, and an intermediate to high affinity to α2- and α3-GABA_{A} receptors, respectively, it is further believed that also as provided by the present invention, the benzodiazepine (Lorazepam) profoundly attenuates anxiety-related symptoms and also prevents prevent nausea and vomiting by preferentially acting the GABA α1 receptor, while also on other subunits, such as α2- and α3-subunits of the GABA_{A} receptors, either individually, or in concert. In fact, such potential actions in concert may be important for the relief of multiple CNS-related disorder for elderly subjects in need of palliative, hospice and other cares, who are likely suffering from one or more of the conditions relating to the subject's old age, which condition is selected from the group consisting of dyspnea (shortness of breath), respiration congestion, agitated delirium, pain (e.g., pleuritic pain, discomfort from immobility and/or pain due to pre-existing conditions), weakness and fatigue, insomnia nausea and vomiting, seizures and poor secretion control.

Based on the above, the present invention, in one of the embodiments, provides a method of relieving multiple CNS-related disorders for elderly subjects in palliative, hospice and other cares, who are likely suffering from one or more of the conditions relating to the subject's old age, which condition is selected from the group consisting of dyspnea (shortness of breath), respiration congestion, agitated delirium, pain (e.g., pleuritic pain, discomfort from immobility and/or pain due to pre-existing conditions), weakness and fatigue, insomnia nausea and vomiting, seizures and poor secretion control and thus, can benefit from a single dose of a benzodiazepine in a quick and sustained-release formulation, which method comprises applying the dual-release flexible thin film formulation of the present invention to the mucosal tissue of the subject, wherein delivered to said subject through the use of the dual-release flexible thin film formulation is a therapeutically effective amount of a composition comprising a compound and a pharmaceutically acceptable carrier, wherein said compound is a positive allosteric modulator of the GABA_{A} receptor, or a pharmaceutically acceptable salt thereof, that is receptor subunit-selective. The selectivity can be function, or affinity-based, or both; and can be towards, primarily, an α1 subunit, an α2, or an α3 subunit, or a combination thereof, of GABA_{A} receptor.

Based upon the aforementioned difference in structures, while similarity in binding sites between Lorazepam and the benzodiazepines, it is also contemplated in other embodiments of the present invention that other compounds of the benzodiazepine family having their mechanisms of actions similar to Lorazepam, particularly those that are full agonists or partial agonists at the α1, α2, or α3 subunits of the GABA_{A} receptors, can also be used with the methods described in the present application. In some embodiments, the benzodiazepine drug is a typical benzodiazepine class compound. In other embodiments, the benzodiazepine drug can be an atypical benzodiazepine class compound. In some embodiments, the benzodiazepine drug acts on the α1-subtype of the GABA_{A} receptor (e.g., has a functional selectivity at the α1 subunit). In other embodiments, the benzodiazepine drug is selective for the α1 subunit, as well as other subunits, such as the α2 and/or α3 subunits, etc., of the GABA_{A} receptor.

A further aspect of the invention is the use of Lorazepam, or an alternative compound, as aforementioned, and optionally in combination with another subunit-selective GABA_{A} receptor agonist, for the manufacture of dual-release flexible thin film formulation of the present invention for elderly subjects in palliative, hospice and other cares.

A further aspect of the invention is a method for treating elderly subjects in palliative, hospice and other cares, whereby a pharmaceutically and pharmacologically effective amount of Lorazepam, or an alternative compound, as aforementioned, and optionally in combination with another subunit-selective GABA_{A} receptor agonist, is administered in a dual-release flexible thin film formulation of the present invention to a subject in need.

### EXAMPLES

In the following examples of Application of Oral Thin Films in Oral Cavity

1. For quick releasing film, the film will dissolve in oral cavity (buccal site or on the tongue) in 20 minutes, and all the drug molecules in the film (0.5 mg or 2 mg) will be released within the 20-minute time frame.

Examples 1 and 2 provide the compositions and detail the preparation method for 0.5 mg and 2 mg lorazepam quick releasing oral films.

2. For slow release film, the film will erode in about 5 hours in oral cavity, and the drug will release slowly as the film erodes, at the rate of about 0.1 to 0.3 mg/h, and in 5 hours the drug will be cumulatively released to 1 mg of drug.

Example 3 provides the composition and details the preparation method for the 1 mg lorazepam slow (sustained) releasing buccal film.

3. Combination of quick and slow release film can be prepared by lamination of the quick and slow releasing films to form a two-layer film. When the 2-layered film is applied onto the oral cavity, the drug release profile will be first, a burst release of 0.5 mg in the first 15-20 min (from the quick-releasing layer), then release the drug from the slow releasing layer at the rate of about 0.1-0.3 mg/h for the next 5 hours, cumulative to 1 mg in 5 hours.

Example 4 describes the preparation of 2-layered films with combined property of burst and sustained drug release.

### Example 1

Dosage Form 1, with a Composition as Shown in Table 1, was Prepared as Follows:

In a 250 mL beaker, 2.7 grams of L-menthol (a flavoring agent) was dissolved in ethanol (˜60 mL). Into the beaker, propylene glycol, water, and peppermint oil were added and stirred for 10 min at room temperature. The solid materials: saccharin, PolyOx N10, and lorazepam were added in sequence while the mixture was being stirred. The stirring of the mixture was continued for 30 minutes till all solid materials were dissolved. The solution was casted using a casting application with 30 mils gap on a polyester release liner (i.e. 3M's 1022 release liner). The coating was allowed to dry in air for 20 min and further in a force air oven for 10 min at 65° C. The dried film was de-laminated from the release liner (the supporting release liner was discarded), and the resulting films were die-cut into a single unit dose of 1 in×1 (25 mm×25 mm) in square article, which contains about 0.5 mg of lorazepam. The cut-die film was put into a 2 in×2 in (50 mm×50 mm) pouch, and the pouch was sealed with a heat sealer.

The batch size for this example was 140 grams. The final dried film was in the form of one inch by one inch square film, with the total weight of 100 mg, containing 0.5 mg of lorazepam. Each film was contained individually in a single-unit pouch.

TABLE 1
Composition and Formulation for 0.5 mg Lorazepam Oral Film

| | | | | | mg |
|---|---|---|---|---|---|
| | | Wet | Dry | | in one |
| Ingredient | Function | grams | Grams | Dry % | film |
| Propylene | Film Plasticizer | 7.65 | 7.65 | 15.30% | 15.30 |
| Glycol | | | | | |
| Lorazepam | Active | 0.25 | 0.25 | 0.50% | 0.50 |
| | Ingredient | | | | |
| Water | Casting solvent | 30 | | | |
| Ethanol | Casting solvent | 60 | | | |
| PolyOx N10* | Film Matrix | 37.00 | 37.00 | 74.00% | 74.00 |
| | Polymer | | | | |
| Saccharin | Sweetening | 0.70 | 0.70 | 1.40% | 1.40 |
| | Agent | | | | |
| L-Menthol | Flavoring | 2.70 | 2.70 | 5.40% | 5.40 |
| | Agent | | | | |
| Peppermint oil | Flavoring | 1.70 | 1.70 | 3.40% | 3.40 |
| | Agent | | | | |
| Total | | 140.00 | 50.00 | 100% | 100.00 |

*Note: PolyOx N10 is nonionic poly (ethylene oxide) polymer with molecular weight of approximately 100,000, manufactured and supplied by Dow Chemical.

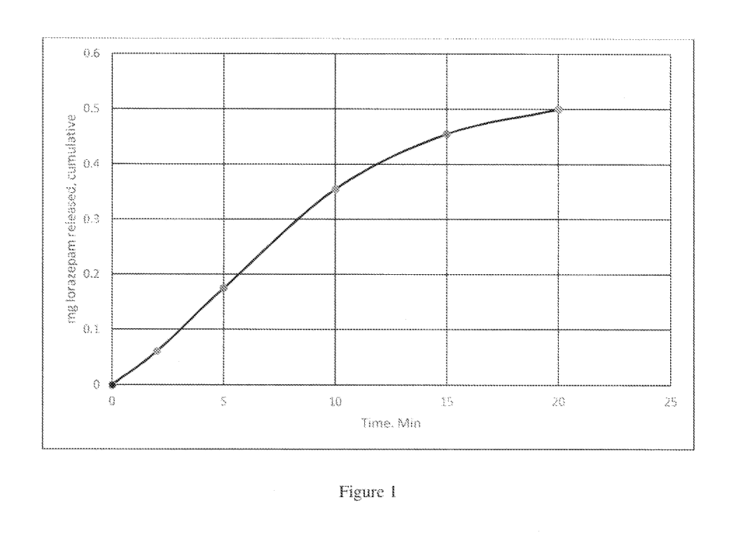

The prepared dosage form of 1 in×1 in (25 mm×25 mm) film was tested for drug release property. This was done by putting one film in a flask with 50 mL phosphate buffer solution of pH 7.4, with light agitation at 50 rpm, in a 37° C. incubator. Samples were taken periodically and analyzed for lorazepam content, using a high performance liquid chromatography (HPLC). Results of the drug release test are presented in FIG. 1.

### Example 2

Dosage form 2 (2 mg lorazepam quick dissolving oral film), with a composition as shown in Table 2, was prepared with a method of similar process steps given in Example 1, wherein the batch size was 140 grams. The final dried film was in the form of 1 in×1 in (25 mm×25 mm) square film, with the total weight of 100 mg, containing 2.0 mg of lorazepam. Each film was contained individually in a sealed single-unit pouch.

TABLE 2
Composition and Formulation for 2 mg Lorazepam Oral Film

| | | | | | mg in |
|---|---|---|---|---|---|
| | | Wet | Dry | | one |
| Ingredient | Function | grams | Grams | Dry % | film |
| Propylene | Film Plasticizer | 7 | 7 | 14.00% | 14 |
| Glycol | | | | | |
| Lorazepam | Active Ingredient | 1 | 1 | 2.00% | 2 |
| Water | Casting solvent | 30 | | | |
| Ethanol | Casting solvent | 60 | | | |
| PolyOx N10 | Film Matrix | 37 | 37 | 74.00% | 74 |
| | Polymer | | | | |
| Saccharin | Sweetening Agent | 0.7 | 0.7 | 1.40% | 1.4 |
| L-Menthol | Flavoring Agent | 2.7 | 2.7 | 5.40% | 5.4 |
| Peppermint oil | Flavoring Agent | 1.6 | 1.6 | 3.20% | 3.2 |
| Total | | 140 | 50 | 100% | 100 |

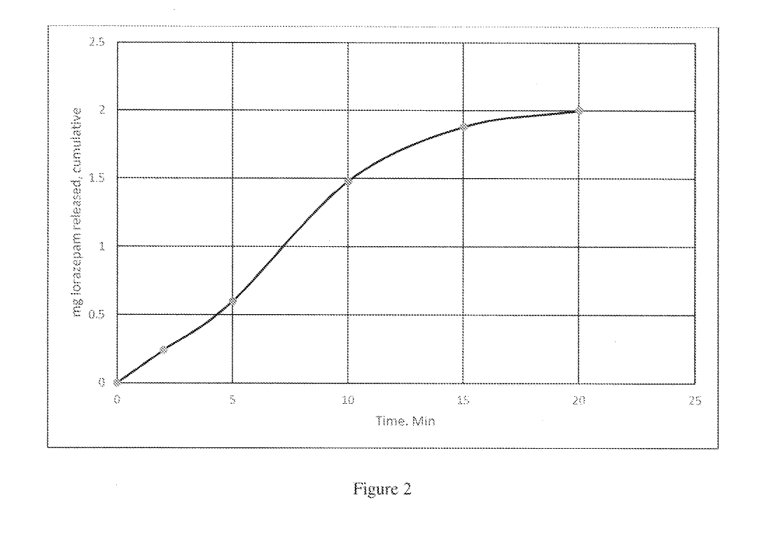

For Dosage form 2, the In vitro drug release testing was performed by using the same protocol as that in Example 1, and results of the testing are shown in FIG. 2.

### Example 3

Dosage form 3 (1 mg lorazepam prolong/sustained-release oral film) with a composition as shown in Table 3 was prepared with a procedure of several process steps.

TABLE 3
Composition and Formulation for 1 mg Lorazepam Slow
Releasing Oral Film

| | | wet | dry | Unit Dose/ |
|---|---|---|---|---|
| Ingredient | Function | grams | grams | 100 mg |
| Lorazepam | Active ingredient | 1 | 1.0 | 1.00 |
| Propylene | Film Plasticizer | 24 | 24.0 | 24.00 |
| Glycol | | | | |
| PEG 400 | Film Plasticizer | 14 | 14.0 | 14.00 |
| L-Menthol | Flavoring Agent | 5 | 5.0 | 5.00 |
| Peppermint oil | Flavoring Agent | 3 | 3.0 | 3.00 |
| Acetone | Solvent | 80 | | |
| Ethanol | Solvent | 80 | | |
| Purified Water | Solvent | 140 | | |
| HPMC E5 | Film Matrix Polymer | 21 | 21.0 | 21.00 |
| CMC Type 12 | Mucosal Bio-adhesive | 32 | 32.0 | 32.00 |
| Total | | 400.00 | 100.0 | 100.00 |

First, an aqueous phase solution was prepared by dissolving hydroxyl methyl cellulose (HPMC) Grade E5, in purified water. Separately, an alcoholic phase was prepared by dissolving L-menthol, propylene glycol, PEG (polyethylene glycol) 400, in an ethanol-acetone mixed solvent. Then, carboxyl methylcellulose (CMC) Type 12 was mixed and dispersed in the ethanol-acetone mixture till the CMC was completed wetted. Finally, the aqueous phase was mixed with the alcoholic phase under high speed stirring of 18,000 rpm for about 1 to 1.5 hours to produce a viscous solution. The resulting solution was casted on a release liner (supporting substrate), air dried for an hour, followed by oven drying at 60° C. for 10 min to produce the master dried matrix film. The release liner was then removed and discarded. The de-laminated master film was die-cut into 1 in×1 in (25 mm×25 mm) square disc, and was individually packaged in a sealed pouch. The final dried film was in the form of 1 in×1 in (25 mm×25 mm) square film, with the total weight of about 100 mg, containing 1.0 mg of lorazepam. Each film was contained individually in a sealed single-unit pouch.

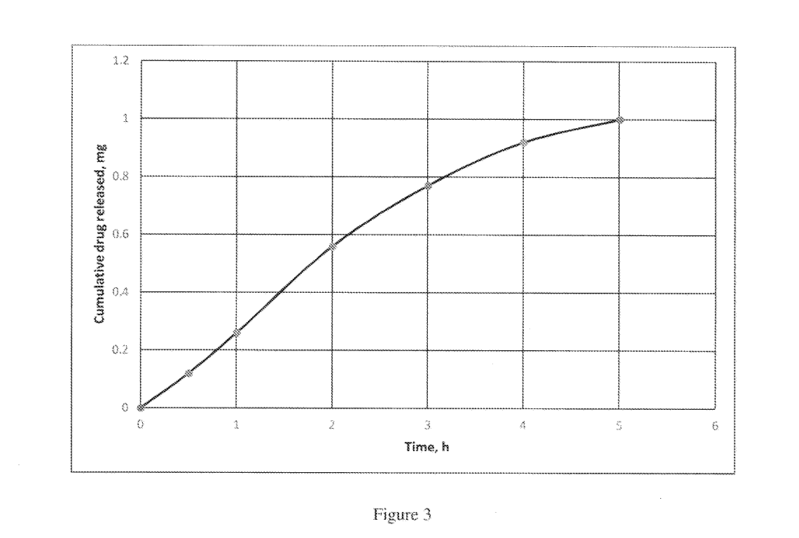

For Dosage form 3, the In vitro drug release testing was performed by using the same protocol as that in Example 1. Results of drug release testing are shown in FIG. 3.

### Example 4

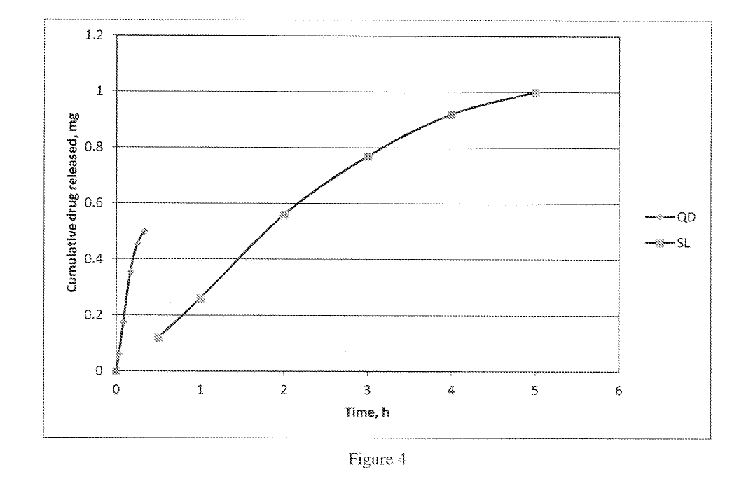

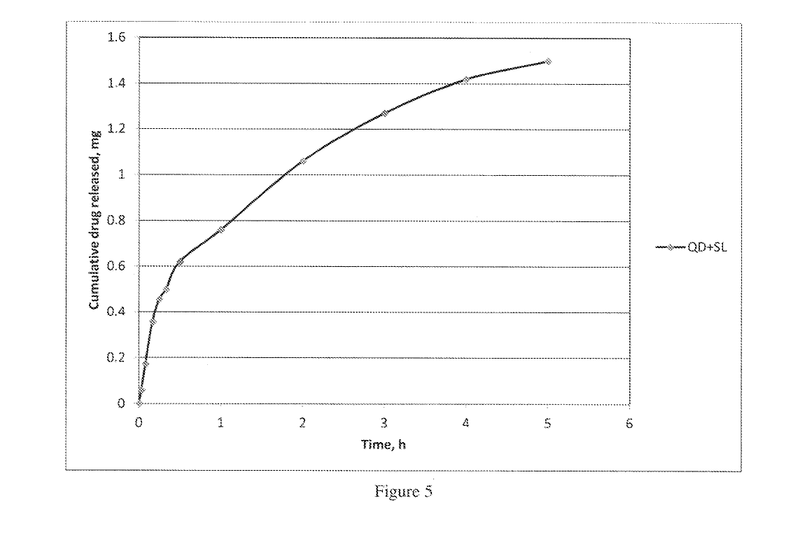

This example illustrates the preparation of lamination (combination) of quick-releasing (QR) film and slow releasing (SR) film to produce a two-layered matrix film with additive drug release profile shown in FIGS. 4 and 5. Dosage form 4 of the combined (laminated) 2-layered matrix film was prepared by first using a procedure of several process steps as described in Example 3 to prepare the master film of sustained-releasing film. Separately, the casting solution of quick releasing (QR) film was prepared by using a procedure as described in Example 1. Next, the QR casting solution thus prepared was casted onto the dried master film of the sustained-releasing film, and the resulting film was dried with a drying procedure as described in Example 1, resulting in a two-layered (QR-SL) film. The master film thus prepared can be die-cut into 1 in by 1 in (25 mm×25 mm) squares and individually pouched, accordingly.

For Dosage form 4, the In vitro drug release testing was performed by using the same protocol as that in Example 1. Results of drug release testing are presented in FIG. 4, which shows the individual drug release profiles of QR and SR films, and in FIG. 5, which shows the expected combined effects on the drug release profile.

The many elements of the present invention make it unique in the field. The novelty is illustrated by the various options for nearly every aspect of the invention that allow it to be used in the proper exercise form by a variety of users, both in terms of body size and fitness level. Additionally, there is a wide range of exercises available to any user of the present invention, and users can perform exercises that use the upper and lower extremity muscle groups simultaneously.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A dual-release, dual-adhesiveness flexible thin film formulation comprising:
a benzodiazepine;
a quick-release subfilm comprising an admixture of polymer, which comprises predominantly highly water-soluble polymer(s), and said benzodiazepine in a first form, the polymers and other components of the quick-release subfilm adapted to provide 20 minute or less erodibility for the quick-release subfilm; and
laminated against the quick-release subfilm, a sustained-release subfilm comprising an admixture of polymer, which comprises predominantly hydrophilic, bioadhesive polymer(s), and said benzodiazepine in a second form, which can be the same as the first, the polymers and other components of the sustained-release subfilm adapted to provide erodibility of 1 hours or more and 20 hours or less for the sustained-release subfilm, said thin film formulation of said benzodiazepine having a thickness of 30 mil or less, and being more adhesive on the sustained-release side.

2. The thin film formulation of claim 1, wherein the highly water-soluble polymers comprise soluble cellulose derivatives, polyvinylpyrrolidone, polyvinyl alcohol, polyethylene oxide, polyvinyl alcohol grafted polyethylene glycol copolymer, or mixtures thereof.

3. The thin film formulation of claim 2, wherein the highly water-soluble polymers comprise soluble polyethylene oxide, polyvinyl alcohol grafted polyethylene glycol copolymer, or mixtures thereof.

4. The thin film formulation of claim 1, wherein the predominantly hydrophilic, bioadhesive polymers comprise xanthan gum, carrageenan, pectin, sodium carboxymethylcellulose, alginate, polyacrylic acids, high molecular weight polyethylene oxide, or mixtures thereof.

5. The thin film formulation of claim 1, wherein the polymers and other components of the quick-release subfilm adapted to provide 15 minute or less erodibility for the quick-release subfilm.

6. The thin film formulation of claim 1, wherein the polymers and other components of the quick-release subfilm are adapted to provide 10 minute or less erodibility for the quick-release subfilm.

7. The thin film formulation of claim 1, wherein the polymers and other components of the sustained-release subfilm are adapted to provide bioactive agent release period of 2 hours or more and 20 hours or less for the bioactive agent provided by the sustained-release subfilm.

8. The thin film formulation of a benzodiazepine according to claim 1, wherein the benzodiazepine is a sub-unit selective positive allosteric modulator (PAM) of the GABA_{A} receptor in the central nervous system (CNS) and is selected from the group consisting of lorazepam, diazepam, alprazolam, estazolam, temazepam, chlordiazepoxide, clonazepam, diazepam, flurazepam, quazepam, clorazepate, oxazepam, midazolam, and triazolam or pharmaceutically acceptable salts thereof.

9. A method of delivering a benzodiazepine that functions as sub-unit selective PAM of the GABA_{A} receptor in the CNS comprises applying the sustained-release side of the thin film formulation of claim 1 to the mucosal tissue of an elderly subject in need of palliative, hospice and other cares.

10. The method of claim 9, wherein the benzodiazepine formulation is applied to the mucosal tissue of the subject's mouth.

11. The method of claim 9, wherein a quick-release side of the benzodiazepine formulation is applied to the top of the tongue, and the formulation is applied by a transfer from the tongue to the palate of the subject.

12. The method of claim 9, further comprising the step of: providing the benzodiazepine formulation so that the quick-release and the sustained release sides have distinct colors, applying the quick-release colored side to a transfer intermediate, and then transferring the formulation to the mucosal tissue of the subject.

13. A flexible thin film formulation for delivering of a benzodiazepine consisting of a sustained-release film which comprises:
a benzodiazepine;
an admixture of polymers to be admixed with said benzodiazepine, comprising predominantly hydrophilic, bioadhesive polymer(s), and the polymers and other components of the sustained-release subfilm adapted to provide a bioactive release period of 1.5 hours or more and 15 hours or less,
wherein the thin film formulation of the benzodiazepine having a thickness of 30 mil or less; and
wherein a predominant amount of the predominantly hydrophilic bioadhesive polymer(s) comprises cross-linked polymers of acrylic acid.

14. The thin film formulation of claim 2, wherein the predominantly hydrophilic, bioadhesive polymers comprise xanthan gum, carrageenan, pectin, sodium carboxymethylcellulose, alginate, polyacrylic acids, high molecular weight polyethylene oxide, or mixtures thereof.

15. The thin film formulation of claim 3, wherein the predominantly hydrophilic, bioadhesive polymers comprise xanthan gum, carrageenan, pectin, sodium carboxymethylcellulose, alginate, polyacrylic acids, high molecular weight polyethylene oxide, or mixtures thereof.

16. The thin film formulation of claim 4, wherein the polymers and other components of the quick-release subfilm adapted to provide 15 minute or less erodibility for the quick-release subfilm.

17. The thin film formulation of claim 17, wherein the polymers and other components of the quick-release subfilm are adapted to provide erodibility in 15 minutes or less for the quick-release subfilm.

18. The method of claim 9, wherein the benzodiazepine is a sub-unit selective positive allosteric modulator (PAM) of the GABA_{A} receptor in the central nervous system (CNS) and is selected from the group consisting of lorazepam, diazepam, alprazolam, estazolam, temazepam, chlordiazepoxide, clonazepam, diazepam, flurazepam, quazepam, clorazepate, oxazepam, midazolam, and triazolam or pharmaceutically acceptable salts thereof.

19. The method of claim 9, wherein the subject is suffering from one or more of the conditions selected from the group consisting of: dyspnea, respiration congestion, agitated delirium, pain, weakness and/or fatigue, insomnia, nausea and/or vomiting, seizures, and poor secretion control.
